(19) United States
(12) Patent Application Publication
(10) Pub. No.: US 2011/0014694 A1
(43) Pub. Date: Jan. 20, 2011

(54) USE OF ENTERIC GLIA

(75) Inventors: Shucui Jiang, Hamilton (CA); Michel Rathbone, Hamilton (CA)

Correspondence Address: BERESKIN AND PARR LLP/S.E.N.C.R.L., s.r.l., 40 KING STREET WEST, BOX 401, TORONTO, ON, M5H 3Y2, CA

(73) Assignee: NEUROLOGICAL TECHNOLOGIES INC., Sutton, ON (CA)

(21) Appl. No.: 12/934,349

(22) PCT Filed: Apr. 1, 2009
(86) PCT No.: PCT/CA09/00412

§ 371 Date: Sep. 24, 2010

Related U.S. Application Data
(60) Provisional application No. 61/041,334, filed on Apr. 1, 2008.

(51) Int. Cl. C12N 5/071

(52) U.S. Cl. 435/366; 435/325

(57) ABSTRACT

Methods of reducing tissue damage in the nervous system are disclosed. The methods involved administering enteric glial cells to an animal with a nerve injury. Methods of improving locomotor function in animal with a nerve injury are also disclosed.

### FIELD

The present application relates to the use of enteric glia (EG) to reduce tissue damage in the nervous system. The application also relates to the use of EG to improve locomotor function in an animal with a nerve injury.

### BACKGROUND

Traumatic injury to the adult central nervous system (CNS) is associated with different types of damage that induce multicellular responses and the formation of a nonpermissive glial environment that inhibits axonal regeneration (Bunge et al., 1997; Schwab, 2000; Bundesen et al., 2003; Lu et al., 2007). After spinal cord injury (SCI), a major pathological feature is the severing of large numbers of nerve fibers (axons) so that communication is interrupted in the long ascending and descending pathways responsible for normal motor, sensory and autonomic functions. The injured axons are prevented from regenerating by an inhibitory environment created by glial scarring, myelin debris and the accumulation of a variety of inflammatory cell types. Over time, a fluid-filled cyst can develop at the site of injury, forming a physical barrier to regeneration (Bunge et al., 1997; Schwab, 2000; Filbin, 2003; Silver and Miller, 2004). These events occur in a delayed yet progressive fashion, and they result in an area of tissue destruction that can extend several segments below the original injury (Dumont et al., 2001). If the injury is incomplete, spared fibers around the lesion can provide some residual function.

In light of these pathological findings, there are several areas that can potentially be targeted in the treatment of SCI. These include reducing secondary damage, enhancing the function of spared pathways and promoting regeneration of the crushed or severed fibers through the unfavorable milieu created by scarring and cyst formation. One approach has been to transplant cells into injury sites in experimental animals with SCI. The cells chosen for transplantation have been either immature cells such as neural stem cells and fetal tissue or glial cells from regions of nervous system that naturally support neuronal activities (Reier, 2004; Barrett and Riddell, 2007).

Since enteric glia cells (EG) play an very important role in the maintenance of tissue integrity and the modulation of neuronal activities in the gastrointestinal tract (Cabarrocas et al., 2003; Ruhl, 2005; von Boyen al., 2006b; von Boyen and Steinkamp, 2006; Vasina et al., 2006) and share morphological, structural and functional properties with astrocytes of the central nervous system (Ferri et al., 1982; Jessen and Mirsky, 1983; Cabarrocas et al., 2003; Vasina et al., 2006) as well as sharing some properties with olfactory ensheathing glia (Barber and Lindsay, 1982; Doucette, 1990; Pixley, 1992), they are a particularly interesting source of material for transplantation into the injured central nervous system (CNS). Furthermore, EG are theoretically available in large quantities, since they can be obtained from the patient's own intestine, conferring the added advantage of circumventing problems of immune rejection following transplantation.

Other investigators have transplanted either myenteric plexus or myenteric ganglia into various areas of the brain or spinal cord (Jaeger et al., 1993; Lawrence et al., 1991; Tew et al., 1992, 1993, 1994) and have demonstrated that axonal sprouts from the host CNS either grow into, or around, those grafts (Jaeger et al., 1993; Lawrence et al., 1991; Tew et al., 1992, 1994). Although it is unclear whether enteric neurons, glia, or smooth muscle cells were responsible for the ingrowth of axons into the mixed grafts of enteric tissues in the studies in which either myenteric plexus or isolated enteric ganglia were implanted into the CNS, the possibility was raised that EG may play very important role in axonal ingrowth and sprouting (Tew et al., 1994).

The present inventors have shown that purified adult EG facilitate ingrowth of transected dorsal root axons into, and through, the spinal cord toward their previous targets (Jiang et al., 2003a). More importantly, the inventors showed that the regeneration of axons induced by the transplantation of EG was accompanied by functional recovery as determined by the cutaneous trunci muscle (CTM) reflex (Jiang et al., 2003b). However, there is still a need to reduce tissue damage at the injury site and to improve locomotor function in spinal cord injuries.

### SUMMARY

The present inventors have administered and enteric glia (EG) to rats with a spinal cord injury and have shown a remarkable reduction in tissue damage at the injury site. Importantly, rats treated with EG did not have cystic lesions although untreated rats did. Cystic lesions are common in spinal cord injuries and can form a physical barrier to regeneration and can extend several segments below the injury.

Accordingly, the application relates to a method of reducing tissue damage in the nervous system comprising administering an effective amount of an enteric glial cell to an animal in need thereof.

The inventors have also shown that rats treated with enteric glia had better locomotor function than control rats.

Accordingly, the present application also provides a method of improving locomotor function in an animal with a nerve injury comprising administering an effective amount of an enteric glial cell to an animal in need thereof.

In a preferred embodiment, the animal with the nerve injury is a human with a spinal cord injury.

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

The application will now be described in relation to the drawings in which:

FIG. 1. Open field walking Test (OFWT) scores from the day prior to transplantation to 8 weeks after transplantation for groups of rats that had received a spinal cord crush injury either and one week later had been injected with medium, with EG or that were not injected (means±SEM; *p<0.05).

FIG. 2. Hind Limb Placing Response (HLPR) scores from the day prior to transplantation to 8 weeks after transplantation for groups of rats that had received a spinal cord crush injury either and one week later had been injected with medium, with EG or that were not injected (means±SEM; *p<0.05). Animals with normal spinal cord function score 2, whereas a score of 0 represents total paralysis.

FIG. 3. Foot orienting response (FOR) scores from the day prior to transplantation to 8 weeks after transplantation for groups of rats that had received a spinal cord crush injury either and one week later had been injected with medium, with EG or that were not injected (means±SEM; *p<0.05). Animals with normal spinal cord function score 2, whereas a score of 0 represents total paralysis.

FIG. 4. Inclined plane test (IPT) scores from the day prior to transplantation to 8 weeks after transplantation for groups of rats that had received a spinal cord crush injury either and one week later had been injected with medium, with EG or that were not injected (means±SEM; *p<0.05).

FIG. 5. Photomicrographs of sagittal sections of thoracic spinal cords, at the injury site 9 weeks post-injury (8 weeks post implantation), stained with hematoxylin and eosin. (A) A cystic cavity is present at the lesion site in a non-EG vehicle subject (medium-injected cord). Boxed region of panel (A) is seen at higher magnification in panel (B). (C) EG-grafted cord exhibit filling of cells and fibers in the lesion site. Boxed region of panel (C) is seen at higher magnification in panel (D) and reveals the cellular and fiber filling in the lesion site. (E) The cystic cavity was significantly smaller in the animals injected with EG compared with animals that received medium only (E; *p<0.05). Scale bar in c=100 μm for a and c; scale bar in d=50 μm for b and d.

FIG. 6. In a spinal cord that had been injected with medium and not with EG, most axons are excluded from lesion site by glial scarring 9 weeks post-injury. (A) Sagittal section 9 weeks post-injury demonstrates a dense halo of GFAP immunoreactivity surrounding lesion site. Boxed region of panel (A) is seen at higher magnification in panel (B). (C) Neurofilament (NF) immunolabeling at the lesion size and boxed region of panel (C) is seen at higher magnification in panel (D) to reveals exclusion of the vast majority of axons from the lesion cavity by gliotic scarring. (E, F) Merger of the two images demonstrates GFAP (green) and NF (red) double fluorescent immunolabeling at lesion site. Scale bar in A=200 μm for A, C, E and in B=50 μm for B, D, F

FIG. 7. In EG-implanted cords, a chronic glial scar surrounds the lesion site but does not block axon penetration 9 weeks after initial injury (8 weeks after implantation). (A) Sagittal section demonstrates a chronic dense GFAP immunoreactivity surrounding lesion site. Boxed region of panel (A) is seen at higher magnification in panel (B). (C) Neurofilament (NF) immunolabeling demonstrates penetration of axons into the lesion size and boxed region of panel (C) is seen at higher magnification in panel (D) to reveals axons in the lesion site. (E, F) Merger of the two images demonstrates GFAP (green) and NF (red) double fluorescent immunolabeling demonstrates ability of axons to penetrate extensive astrocytic scar and grow into lesion site. Scale bar in A=200 μm for A, C, E and in B=50 μm for B, D, F.

FIG. 8. EG transplantation promoted the outgrowth of growth-associated protein-43 (GAP-43)-positive fibers in the injured spinal cord. Sagittal sections stained with anti-GAP-43 antibody in the vehicle control animals (A), animals received EG implantation (B, C, D). (a) Shows some GAP-43 immuno-positive axons around injured site. (B) Demonstrates significantly more GAP-43 immuno-positive axons at the injured site in the cord from EG transplanted animal and those sprouting axons grow into the lesion site. (C) Identification of EG in lesion site with Hoechst label. (D) Merger of the two images demonstrates EG company with sprouting axons. Scale bar=50 μm for all.

FIG. 9. EG cells were maintained in standard culture medium containing 1% FBS for 24 hours (as described in Example 1), then cells were deprived of serum for 24 hours before the beginning of the experiment. All experiments were carried out in 2 ml culture medium with 2% FBS per well. One ml of medium was removed at the indicated times and examined for neurotrophic factor concentration by ELISA (see Example 1). EG cells release NGF (A) and BDNF (B) under control conditions, in a time dependent manner, with the levels reaching an average of approximately 100 μg/ml of culture medium after 48 hours. All bars represent the means±SEM of 5 independent experiments.

FIG. 10. Staining of human myenteric and submucosal plexus-derived cells with rabbit anti-GFAP. Secondary antibody was (A, B) donkey anti-rabbit IgG—Alexa 594 or (C, D) omitted as control. A,C fluorescence detected at 560-615 nm. B, D, phase contrast images of areas imaged in A, C, respectively.

FIG. 11. Staining of human taenia coli-derived cells with rabbit anti-GFAP. Secondary antibody was (A, B) donkey anti-rabbit IgG—Alexa 594 or (C, D) omitted as control. A, C fluorescence detected at 560-615 nm. B, D phase contrast images of areas imaged in A, C, respectively.

FIG. 12. Effect of EG-conditioned medium with or without neutralizing antibody to β-NGF on neurite number in dissociated DRG. Black line: unconditioned medium without antibody. Green line: unconditioned medium with antibody. Blue line: conditioned medium with antibody. Red line: conditioned medium without antibody. P values were determined by contrast analysis of a univariate ANOVA.

FIG. 13. Phase-contrast images representing typical areas of wells examined for neurite outgrowth study (see Table 2). Antibody (if added) was goat anti-rat β-NGF at 1 μg/mL. Well surface was coated with poly-L-lysine and laminin unless stated otherwise. A) Without conditioned medium or antibody. B) With conditioned medium, without antibody. C) With conditioned medium, with antibody. D) Without conditioned medium, with antibody. E) Without conditioned medium or antibody; well uncoated. F) With conditioned medium, without antibody, well uncoated.

### DETAILED DESCRIPTION

One week after spinal cord injury in adult Wistar rats, the inventors introduced EG, culture medium without EG, or nothing into the injury site. Behaviour was tested weekly for eight weeks in the three groups of animals, which were then killed by perfusion fixation. Rats that received EG had better locomotor function than either set of control rats. There was also a significant reduction in tissue damage of the spinal cords transplanted with EG compared to the two control groups. Cystic cavities were present two months after injury in spinal cords of both control groups. In contrast, rats injected with EG did not have cystic lesions, the injury site consisted of cellular material and nerve fibres, and axon growth could be seen with dense labeling of neurofilament-positive axons within the injury site. Additionally, growth-associated protein-positive sprouting axons were intimately associated with the transplanted enteric glia. The cultured EG that the inventors used secreted nerve growth factor and brain-derived neurotrophic factor, raising the possibility that EG may enhance morphological and functional improvement at least in part as a result of their ability to release neurotrophic factors after transplantation into injured spinal cords in rats.

In one embodiment, the present application provides a method of reducing tissue damage in the nervous system comprising administering an effective amount of an enteric glial cell to an animal in need thereof. The present application also provides a use of an effective amount of an enteric glial cell to reduce tissue damage in the nervous system. The application further provides a use of an effective amount of an enteric glial cell for the manufacture of a medicament for reducing tissue damage in the nervous system.

The term “enteric glial cell” as used herein means a glial cell obtained from the enteric nervous system. Preferably, the EG cell is a Type II EG cell that has many long processes and has high levels of glial fibrillary acidic protein (GFAP). Preferably, prior to transplantation, the EG cells are purified and cultured in vitro. In one embodiment, the EG cells are obtained from the animal to be treated, purified and cultured in vitro and then re-inserted into the same individual as an autologous transplantation. The EG cells are preferably human and obtained from the human to be treated. The results in Example 2 demonstrate that EG have been successfully obtained from human taenia coli and small intestine.

The term “a cell” as used herein includes a single cell as well as a plurality or population of cells.

The term “effective amount” as used herein means an amount effective at doses and for periods of time necessary to achieve the desired amount, e.g. for reducing tissue damage.

The term “reducing tissue damage” as used herein means that the damage to the tissue in the nervous system in the presence of the EG cells is less than observed in the absence of the EG cells. Reducing tissue damage includes reducing glial scarring and reducing the formation of cystic lesions. The presence of tissue damage can be tested using techniques known in the art. In one embodiment, lesion can be quantified using H&E staining as described in Example 1.

The term “nervous system” as used herein includes both the peripheral nervous system (PNS) and the central nervous system (CNS).

The term “animal” as used herein includes all members of the animal kingdom, including humans. Preferably, the animal to be treated is a human having a condition involving or caused by nerve injury. Examples of nerve injuries include neurotrauma, stroke and cerebral ischemia as well as peripheral nerve injuries or neuropathies of any type, including traction injuries, paralysis and neuropathic (neurogenic) pain syndromes. Most preferably, the person has a CNS or PNS injury.

The animal may also have a neurodegenerative disease that causes tissue injury in the nervous system. Examples of neurodegenerative diseases that may be treated according to the present application include Alzheimer's disease, Parkinson's disease, multiple sclerosis, Huntington's disease, Bell's palsy, Pick's disease and amyotrophic lateral sclerosis.

In a specific embodiment, administration of the EG cells induces or improves axonal ingrowth and sprouting into the injury site. After a spinal cord injury, there is scarring at the injury site that excludes axons from the lesion cavity. The present inventors have shown that treatment with EG cells allows axons to penetrate and regenerate into the glial scar. In particular, the inventors have demonstrated the sprouting of growth associated protein-43 (GAP-43) axons at the lesion site. The GAP-43 positive axons were intimately associated with the EG at the lesion site. Therefore, EG appear to both enhance the growth of regenerating axons and to induce fundamental changes to the sealed astrocyte barrier.

The inventors have also shown that transplantation of EG into spinal cord can improve locomotor function in an animal with a nerve injury. Accordingly, the present application also provides a method of improving locomotor function in an animal with a nerve injury comprising administering an effect amount of an enteric glial cell to an animal in need thereof. The present application also provides a use of an effective amount of an enteric glia cell for of improving locomotor function in an animal with a nerve. The application further provides a use of an effective amount of an enteric glia cell for the manufacture of a medicament for of improving locomotor function in an animal with a nerve injury.

### EXAMPLES

The following non-limiting examples are illustrative of the present application:

### Example 1

### Animals

Adult female Wistar rats (280-300 g body weight, Charles River) were maintained in a temperature-controlled vivarium on a 12:12 h light:dark cycle with food and tap water freely available. Rats were handled daily for 2 weeks before surgery and trained in open field walking (Basso et al., 1995, 1996) and inclined climbing tasks (Rivlin et al., 1977; Bresnahan et al., 1987).

### Isolation and Culture of Enteric Glia

Enteric glia were prepared from segments of rat intestine and grown in culture as previously described (Middlemiss et al., 2002; Jiang et al., 2003a, 2003b, 2005). Briefly, rats were euthanized and the small intestine was removed. Opened segments of intestine were washed several times with phosphate buffered saline (PBS) containing antibiotics and fungizone (Middlemiss et al., 2002). Segments of intestine were treated first with dispase, then with 1% N-acetylcysteine (Middlemiss et al., 2002). The medium was replaced with one containing 20% fetal calf serum, penicillin, streptomycin, insulin, transferrin, sodium selenite, and hydrocortisone (Middlemiss et al., 2002) and then filtered through a nylon mesh with openings of 74 μm. Cells were plated into 6-well tissue culture plates. An insert with a feeder layer of growth-arrested 3T3 Swiss albino mouse embryonic fibroblasts was added to each well of the plate as a source of growth factors (Middlemiss et al., 2002). After 4 weeks, EG were subcultured into 100 mm dishes and were ready for transplantation when they reached confluence.

### Spinal Cord Injury

Before surgery, rats were given buprenorphine (0.03 mg/kg body weight, subcutaneously) for pain relief and then were anesthetized with isoflurane (3-5%): O₂ (1 L/min), and a laminectomy at T11/T12 was performed to expose the spinal cord which was then crushed with modified coverslip forceps (Blight, 1991; Gruner, 1996, Jiang et al., 2003c, 2004). The forceps were closed slowly compressing a 5 mm length of the spinal cord to a thickness of 1.4 mm for 15 seconds. The wound was closed by suturing muscles and fat pad, and clipping the skin with stainless steel clips. Postoperatively, the rats were kept quiet and warm.

Re-Operation and Cell Transplantation into Injured Spinal Cords

A suspension of EG was prelabeled with bisbenzimide, a nuclear fluorochrome (Hoechst 33342, Jiang et al., 2003a, 2003b) immediately before implantation. One week following spinal cord crush, rats were re-operated at the initial injury site leaving the dura intact. Animals were divided into 3 groups. In group 1 (n=10), a suspension of enteric glia (1×10⁵ cells/pi culture medium) was drawn into a siliconized glass pipette (100 μm outer diameter, 80 μm inner diameter) attached to a 5 μL Hamilton syringe. Two μL of cell solution was injected at a rate of 1 μL/minute into the centre of the crush injury. In group 2, control animals (n=11) were injected with fresh culture medium alone. In group 3, animals (n=5) did not receive any injection after laminectomy and served as sham-operated controls. The inventors then compared the results among animals injected with EG, control animals, and sham-operated animals using behavioral and histological analysis.

### Behavioral Assessment

All rats were handled daily for 2 weeks preoperatively to acclimatize them to the handling procedures and behavioral testing. After spinal cord compression, the locomotor behavior and segmental reflexes of the rats were assessed immediately prior to transplantation and then weekly after transplantation until the end of the experiment. Four tests were used (Jiang et al., 2004): an open field walking task (Basso et al., 1995, 1996), hind limb placing response (Gruner et al., 1996), foot orienting response (Gruner et al., 1996; Kerasidis et al., 1987) and an inclined plane test (Rivlin et al., 1977; Bresnahan et al., 1987). All behavioral analyses were performed by individuals who were blinded with respect to treatment.

An open field walking task (OFWT) was conducted in a child's circular plastic swimming pool (1.3 m in diameter, Jiang et al., 2003c, 2004). Cagemates (two animals) were placed in the center of the open field. They were observed for 5 min periods, and scored for general locomotor ability using the standard BBB scale. The rats were rated on a scale of 0 to 21, 0 being no function and 21 being normal. If the animal stopped moving for a minute, it was placed again in the center of the open field; otherwise it was left undisturbed for the duration of the 5-min test period.

Hind limb placing response (HLPR) and foot orienting response (FOR) were each scored on a scale of 0 to 2, 0 indicating no function and 2 indicating full function (Kerasidis et al., 1987; Jiang et al., 2004). Half-scores were assigned if the behavioral response appeared intermediate. Hind limbs were scored separately for each measure. To assess HLPR, the hind foot was grasped between the thumb and forefinger, pulled backwards, and then released. The placement of the foot on the table surface was then scored (Gruner et al., 1996; Jiang et al., 2004). The FOR followed Gruner's (1996) protocol modified from the previous descriptions of this reflex (Kerasidis et al., 1987). When a rat is raised and lowered by the tail, it shows a characteristic behavior of the hind legs. A normal rat spreads the toes of its hind legs wide apart and generally holds them apart for several seconds. After spinal cord injury, this response is sometimes lost completely, or reduced in magnitude.

The inclined plane test (IPT) measured the ability of the rats to maintain their position for 5 s on an inclined plane, covered by a rubber mat containing horizontal ridges (1 mm deep, spaced 3 mm apart). The rats were observed as the angle of the surface was increased from 5° to 90° at 5° intervals. The angle at which the rats could no longer stay in position was the outcome measure.

### Spinal Cord Tissue Processing

Animals were killed 2 months after the time of implantation. All rats were deeply anesthetized with sodium pentobarbital (50-60 mg/kg b/w, i.p.) and perfused transcardially, first with 100 ml 0.05 M PBS containing 0.1% heparin, followed by 300-500 ml of 4% paraformaldehyde (PFA). The T9 to L1 segments of the spinal cords were removed and incubated in the same fixative solution overnight at 4° C. and then cryo-protected in 30% sucrose PBS solution for at least 3 days. A segment of each cord, extending from 5 mm rostral to 5 mm caudal to the lesion site, was embedded in medium (Tissue-Tek® OCT compound, Sakura Finetek USA, Inc., Torrrance, CA90504 USA). Serial sections were cut at 20 μm intervals on a cryostat and mounted onto slides (ColorFrost/Plus; Fisher, Pittsburgh, Pa., USA) for histological and immunohistochemical staining and analysis. Some spinal cords were sectioned transversely, others sagittally. Some sections were stained with hematoxylin and eosin (H&E) and others for immunohistochemical analysis using the antibodies described in the section below.

### Immunohistochemistry

Details of these procedures have been described previously (Jiang et al., 2003c, 2004). Briefly, the cryostat sections were thawed, air dried and then incubated in hydrogen peroxide to reduce endogenous peroxidase activity, before being rinsed in PBS. The sections were then incubated in 1% sodium borohydride for 15 min. After thorough washing with PBS, the sections were treated with PBS/5% normal goat serum with 0.3% Triton X-100 at room temperature for 30 min. Overnight incubation with one of the following primary antibodies was performed in humidified boxes at 4° C.: rabbit anti-glial fibrillary acidic protein (GFAP) polyclonal antibodies (1:600; Zymed® Lab-SA System Kit, Invitrogen Canada Inc. Burlington, ON); mouse monoclonal antibodies to anti-neurofilament (RT-97, 1:50, Vector lab Inc. Burlingame Calif. USA); and polyclonal antibodies to neuronal growth-associated protein (GAP-43, 1:500 RDI Inc. Flanders, N.J. USA). The following day, sections were rinsed with PBS and incubated with either rhodamine-conjugated or fluorescein isothiocyanate (FITC)-conjugated secondary antibodies (Jackson ImmunoResearch Lab Inc. Mississauga ON, Canada). Sections were then rinsed, coverslipped and examined under a confocal microscope. Histological analysis was conducted by an investigator who was blinded to the treatment groups.

### Quantification of Lesion Size

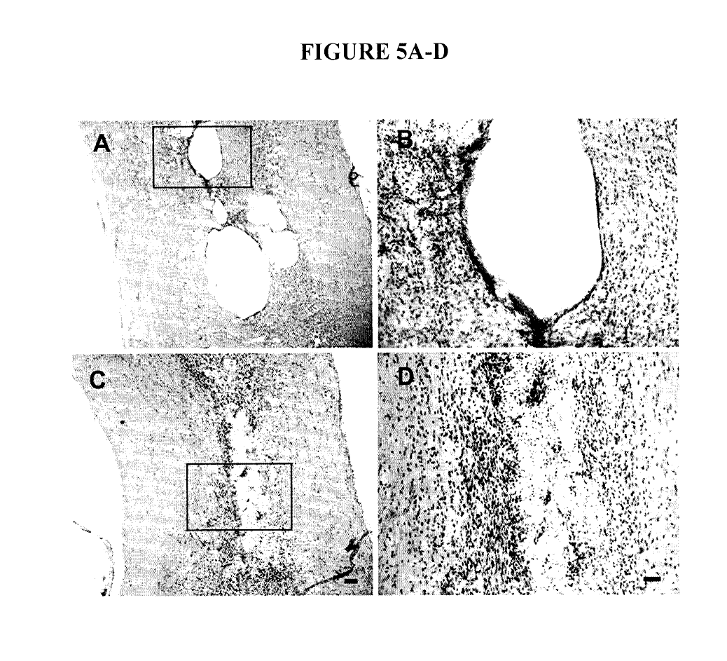

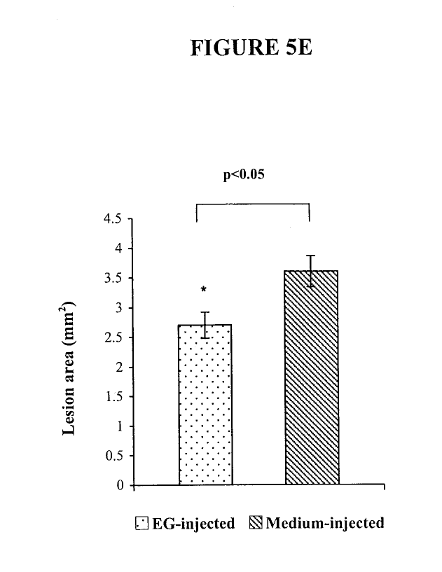

Five sagittal sections (1 at the lesion centre that contained the biggest lesion area, the adjacent 2 dorsal and 2 ventral sections at 100 μm intervals) were taken from each cord and stained with H&E to measure the spinal cord lesion size (Basso et al., 1996; Jiang et al., 2004). The lesion was identified as a cystic cavity delineated by an H&E-positive area (FIG. 5). The average area of the cystic cavity was determined using a Bioquant BQ-TCW98 image analysis program by an investigator who was blind to group assignment (Basso et al., 1996; Jiang et al., 2004).

### Determination of Elaboration of Neurotrophic Factors NGF, NT-3, BDNF and GDNF by Enteric Glia

Enteric glia that had been isolated and grown to confluence (Middlemiss et al., 2002) were trypsinized. After trypsinization, EG cells were plated at a concentration of 200,000 cells per 35 mm well in 6 well plates. The cells were grown in 2 ml of Dulbecco's Modified Eagle Medium (DMEM, Gibco, Invitrogen Canada Inc. Burlington, ON) with 10% fetal bovine serum (FBS, Gibco, Invitrogen Canada Inc. Burlington, ON) and 1% penicillin/streptomycin (Pen/Strep, Gibco, Invitrogen Canada Inc. Burlington, ON) for 24 hours. The cells were then washed in phosphate buffered saline pH 7.4 (PBS) and re-suspended in 2 ml DMEM containing 2% fetal bovine serum and 1% penicillin/streptomycin (Pen/Strep, Gibco, Invitrogen Canada Inc. Burlington, ON) for 24 hours. Then, at 0, 2, 4, 6, 8, 12, 36, and 48 hours the cells were centrifuged and the growth factors were measured in the supernate using commercially available ELISA kits for nerve growth factor (NGF), brain derived neurotrophic factor (BDNF), neurotrophin 3 (NT-3), and glial derived neurotrophic factor (GDNF) (Promega Corp., WS) and using the protocols described by the manufacturer.

### Statistical Analysis:

The statistical significance of behavioral scores was analyzed by Kruskal-Wallis non-parametric analysis of variance followed by Mann-Whitney U tests. Histological data were evaluated by Student's t tests.

### Results

### Behavioral Outcome

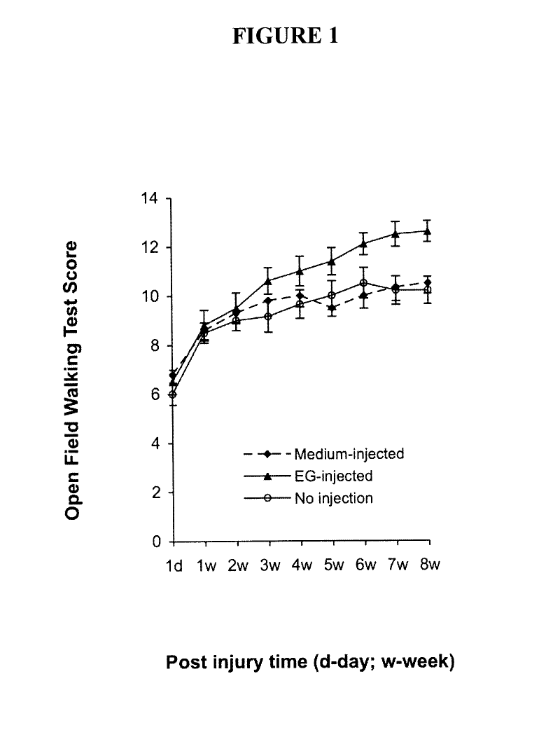

Over the course of 9 weeks, i.e. 1 week after initial spinal cord injury and 8 weeks after re-operation, control rats that received either vehicle (medium) injection or sham-operated rats recovered occasional weight-supported plantar steps, with no fore-hind limb coordination, reflecting a mean BBB locomotor score of about 10 (10.4±0.3 and 10.2±0.5 respectively) (FIG. 1). In contrast, rats that had EG-transplanted into the injury site significantly recovered their locomotor function (P<0.05) and had a BBB score of about 12.6±0.4 (FIG. 1). They exhibited consistent weight supported plantar steps with occasional fore-hind limb coordination.

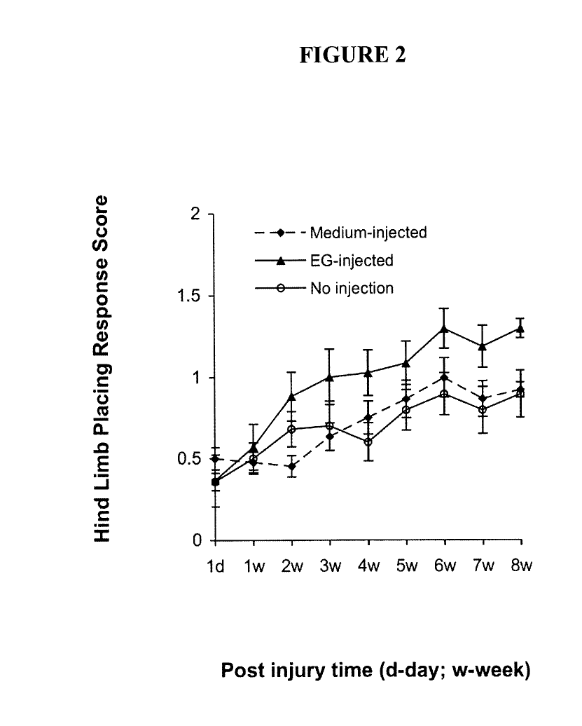

Uninjured rats have normal HLPR scores of 2 (FIG. 2). They always place an extended hind limb briskly beneath the body in a proprioceptive placing response. Injured rats place their hind limb either partially, unreliably, or not all, depending on the time since the injury and the treatment. Nine weeks after injury (8 weeks after re-operation for injection), control rats with either medium injection or no injection attained an HLPR score of 0.9±0.04 and 0.9±0.14 respectively (FIG. 2); there was little or no attempt to place the foot, or the foot remained extended with its dorsal surface facing downward. In contrast, although rats that received EG-injection still had some impaired placement, they had quicker retraction of the limb; the foot was placed with the plantar surface facing downward and, despite having some dragging of dorsal surface/knuckles before placement of the foot and some toe curl, they reached a score of 1.3±0.06 (FIG. 2).

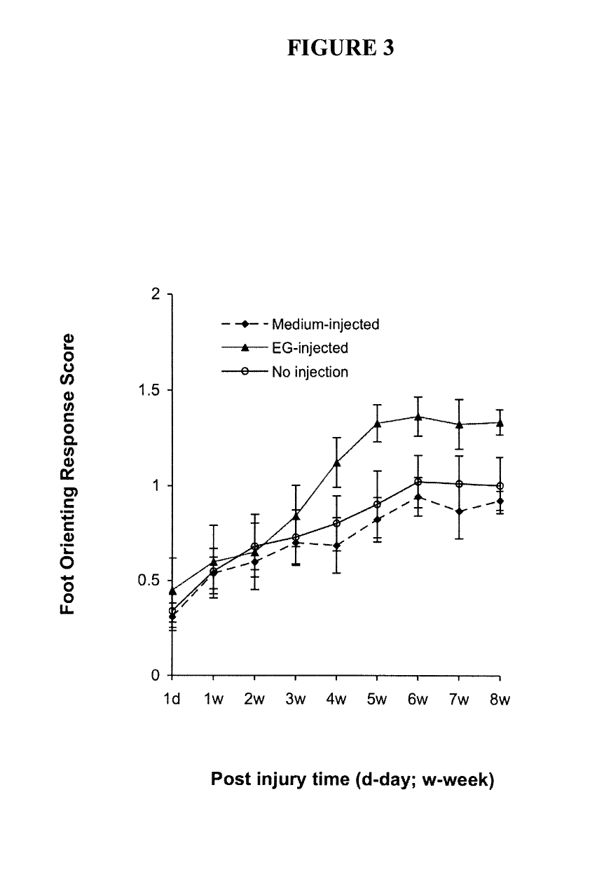

Uninjured rats have a normal FOR score of 2. In these experiments, rats injected with medium or with nothing following spinal cord injury had a FOR score of 0.9±0.05 and 1.0±0.14 respectively (FIG. 3). These rats extended their hind legs laterally with toe spread but turned their feet outward. When these rats were lowered, they did not orient their feet toward the surface. In contrast, the rats injected with EG achieved some recovery. When held suspended by the tail, the hind limbs were spread laterally although at times the hind limbs were spread further apart or closer together than normal and obtained a mean score of 1.3±0.06 (FIG. 3), which is significantly different (p<0.05) from the control rats with no injection or medium-injection.

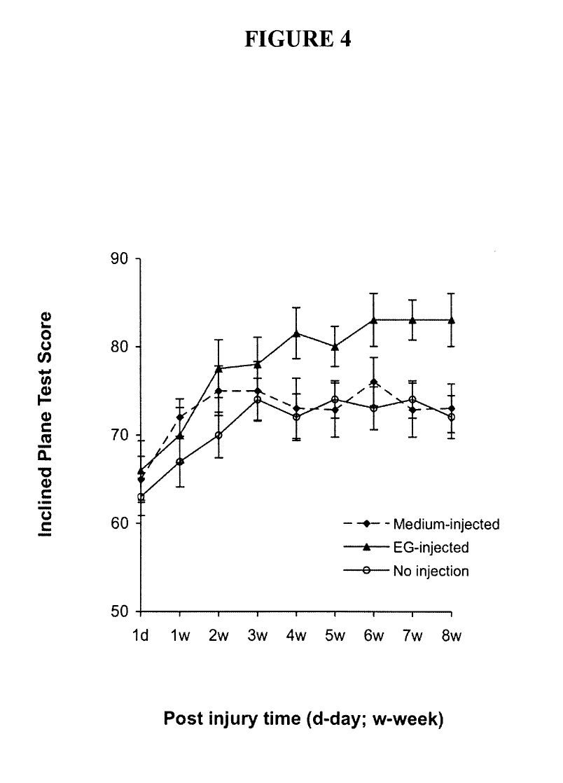

Uninjured rats maintain their position on an inclined plane even at an angle of 90°. After injury, medium-injected or uninjected control rats lost their ability to maintain their position beyond 65°, recovering to 73°±2.7 and 72±2.4 respectively by 2 months after injury. In contrast, rats which had received a transplantation of EG were able to maintain their position to a mean incline of 83°±3.0, which is significantly better (p<0.05) compared to both control groups (FIG. 4).

### EG Implantation Reduces Lesion Size and Fills the Lesion Cavity

At 2 months, all injured cords stained with H&E had obvious tissue damage at the injury site (FIG. 5 A-D). Rats into which no EG had been injected had large cystic cavities at the injury site 9 weeks post-injury (FIG. 5A, B). In contrast, the lesion site was filled with cells and nerve fibres after 2 months in rats that received EG transplantation (FIGS. 5C, D). Consequently, the mean cystic cavity size (4-6 rats per group, 5-7 sections per rat) showed a much reduced cavity size in EG-transplanted rats compared to vehicle controls (medium-injected animals) (FIG. 5E; p<0.05).

Axons are Embedded within a Dense Glial Scar after SCI, and EG Implantation Stimulates Axonal Penetration and Regeneration into the Dense Glial Scar.

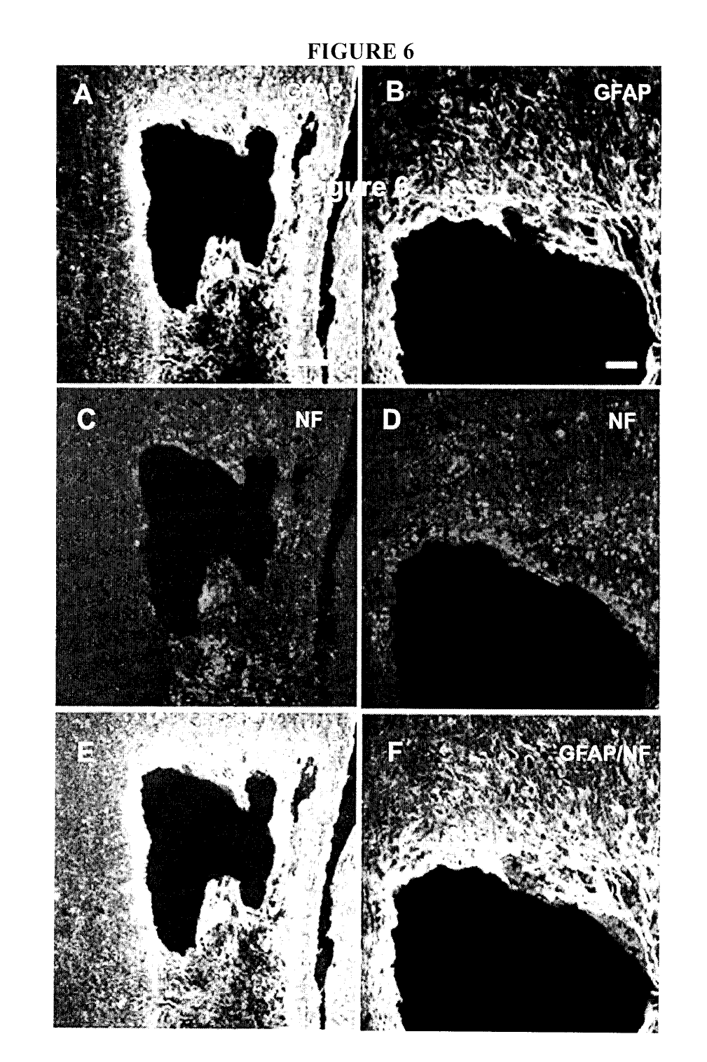

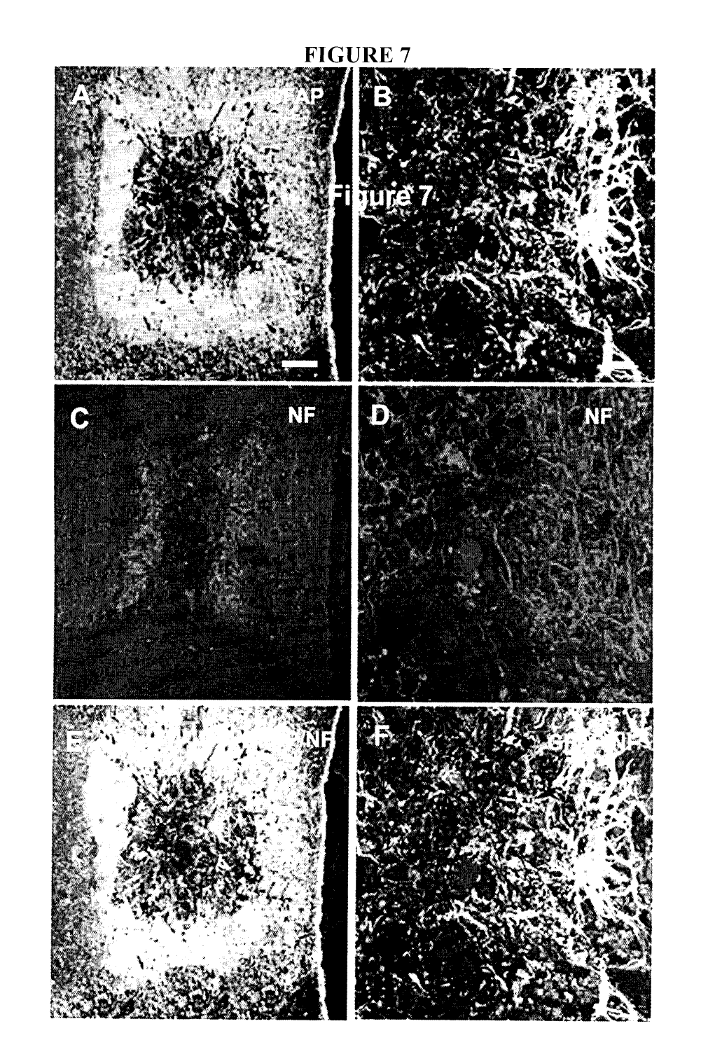

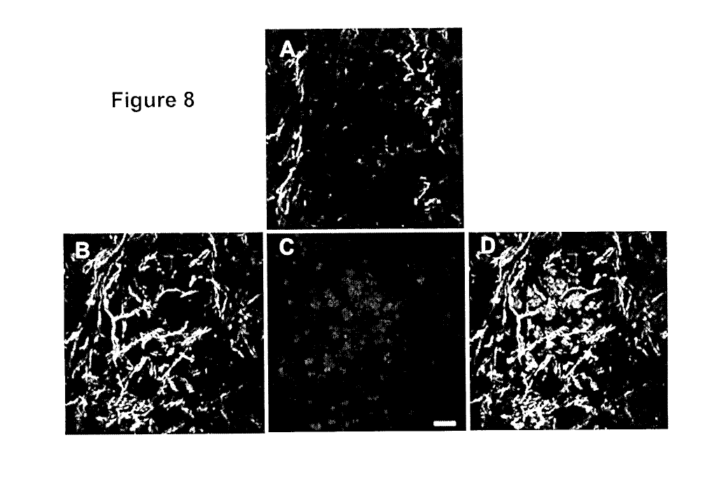

Previous studies report prominent scarring 7 days after SCI and well-formed, persistent gliosis by 14 days in adult rats (Barrett et al., 1984; Frisen et al., 1995) that is still present at 6 weeks after injury (Lu et al., 2007). In the present study, control rats that received only injection of medium 1 week after SCI demonstrated dense GFAP immunoreactivity surrounding the lesion site 9 weeks post-injury (FIG. 6 A, B, E, F). Neurofilament (NF)-immunolabeling demonstrated that, at this time, most axons were excluded from the lesion cavity by this dense scarring (FIG. 6 C, D, E, F). In contrast, despite the persisting, dense GFAP-positive astroglial processes surrounding the lesion cavity (FIG. 7 A, B, E, F), NF-labeled axons were able to cross the dense GFAP boundary and enter the lesion site (FIG. 7 C, D, E, F). Immunoreaction with growth-associated protein (GAP)-43 to determine the effect of implantation of EG on axonal regeneration and sprouting demonstrated that EG prelabeled with Hoechst 33342 (Jiang et al., 2003a, 2003b) accompany newly regenerating, GAP-43-positive axons at the lesion site (FIG. 8).

### EG Release NGF and BDNF

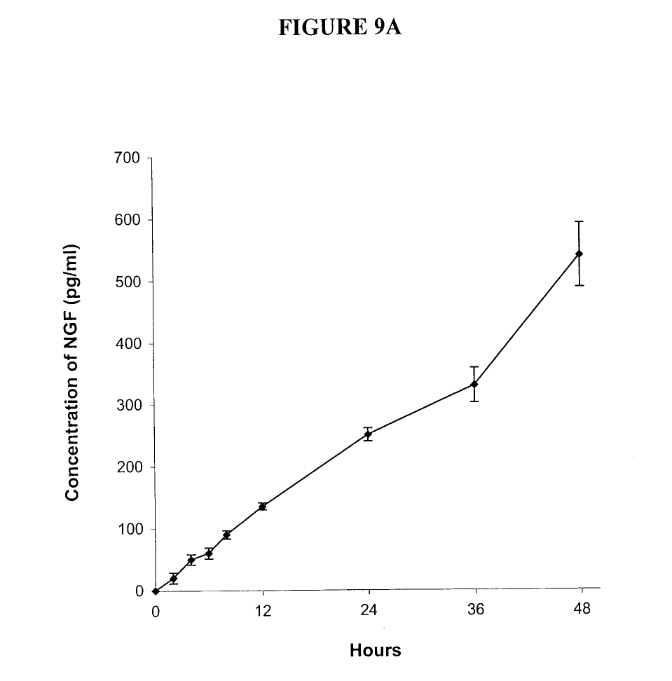

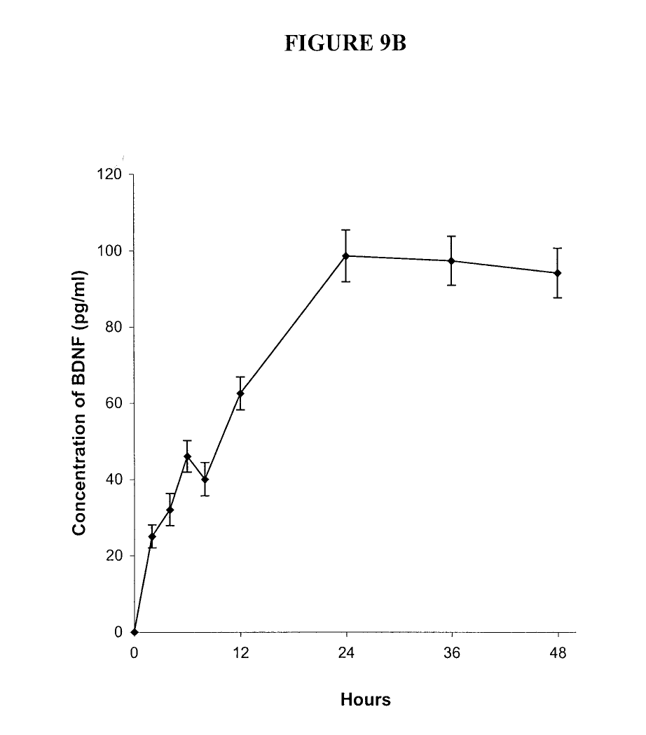

EG cells cultured in vitro release NGF (FIG. 9A) and BDNF (FIG. 9B) under controlled conditions, over time with their respective concentrations reaching approximately 500 μg/ml and 100 μg/ml of culture medium after 48 hours. There was no detectable release of either NT-3 or GDNF from cultured EG cells (data not shown).

### Discussion

The present results indicate that transplantation of EG into spinal cords of rats one week after a crush injury significantly improves functional recovery, reduces spinal cord damage, and stimulates axonal ingrowth and sprouting into the lesion site.

In this example, the ability of rats to walk, place their limbs and maintain their position on an inclined plane was improved after transplantation of enteric glia. The difference in BBB locomotor score between animals after grafting was modest but significant (p<0.05) compared with either medium injection or sham operation (BBB scores 12.6±0.4; 10.4±0.3; 10.2±0.5 respectively), a finding that supports the observations of Pearse et al. (2007) who showed a similar extent of improvement in differences in BBB scores between animals with SCI which had received a combined graft of both olfactory ensheathing glia and Schwann cells (12.3±0.7 cf 10.7±0.7 in controls). Since locomotion and limb placement depend on multiple ascending and descending axonal tracts (Basso, 2000), the improved recovery for all four behavioural measures tested in the group with transplanted EG could be related to the growth of multiple ascending and descending axonal tracts including cortico-, rubro-, reticulo-, vestibulo-, and raphe-spinal tracts.

Surprisingly, the transplantation of EG significantly reduced the extent of tissue damage compared to the spinal cords of control rats into which only medium was transplanted or that received no transplantation. Thus, cellular cystic cavities were found by 2 months after injury in the spinal cords of rats that received no transplantation or that received a transplantation of medium alone. In contrast, EG appeared to become integrated into the spinal cords after transplantation so that the cords from rats injected with EG did not have cystic lesions. The injury sites in spinal cords that were injected with EG consisted of cellular material (mainly astrocytes and enteric glia) and nerve fibres; with the graft area supported axon growth as seen by dense labeling of neurofilament-positive axons within the injury. Additionally, GAP-43 positive sprouting axons were intimately associated with the transplanted enteric glia.

The mechanisms by which EG exert their beneficial effects on locomotor function and on the histological appearance of the lesions are not clear but are likely a result of shifting the balance from the non-permissive environment after injury to one that is permissive. EG are similar in some ways to astroglia of the CNS (Jessen and Mirsky, 1980, 1983; Savidge et al., 2007a), and also appear to have some properties similar to olfactory ensheathing glia (Doucette, 1990; Pixley, 1992; Middlemiss et al., 2002; Jiang et al., 2003a). Since EG lack a complete basal lamina, they can integrate into the CNS after transplantation (Tew et al., 1994; Jiang et al., 2003a, 2003b, 2005). The previous study showed that EG enhance the ability of axons to cross the non-permissive dorsal root entry zone and allow the regenerating dorsal root axons to be directed toward their normal targets (Jiang et al., 2003a, 2003b). In the present study, there was also no apparent barrier at the injury site in cords transplanted with enteric glia, whereas there was a clearly demarcated barrier around the cystic cavities of the control cords. Importantly, the inventors observed regeneration of GAP-43 positive axons that were in close proximity to EG. Thus the EG appear both to enhance the growth of regenerating axons and to induce fundamental changes to the sealed, astrocyte barrier. In future studies, the inventors will investigate whether EG, like olfactory ensheathing glia, form channels through the barrier making pathways through which axons could regenerate (Richter and Roskams, 2007).

Within the CNS, astrocyte end-feet processes come into close contact with cerebral capillaries. As a result, astroglial-derived soluble mediators and extracellular matrix components can contribute the maintenance of blood-brain barrier functions essential for normal function of the brain and spinal cord (Abbott et al., 2006; Bechmann et al., 2007). EG have morphologic and functional similarities to CNS astrocytes (Savidge et al., 2007a) and their processes are in close proximity to gut epithelial cells of the intestinal blood barrier, analogous to the relationship of astrocytes to cerebral endothelial cells. Recent studies have shown that EG secrete several mediators implicated in blood-brain barrier formation (Savidge et al., 2007a, 2007b; Neunlist et al., 2007; Von Boyen et al., 2006a). Several lines of evidence indicate that mediators derived from EG responsible for promoting barrier properties in intestinal epithelia might similarly promote blood-brain barrier characteristics in CNS endothelia (Savidge et al., 2007b). It is particularly interesting that the ability to induce barrier functions is, in many cases, interchangeable among astroglial-like cell types and target cells. For example, the inventors previously reported that transplantation of enteric glia into the spinal cord accelerates normal repair processes of the vasculature at the site of injury and promotes the induction of a functional blood-brain barrier (Jiang et al., 2005). Therefore, EG-induced beneficial effects on locomotor function and on the histological appearance of the lesions may be, at least in part, due to improving blood-brain barrier function and therefore enhancing blood supply to the injured tissue.

EG are capable of producing a number of neurotrophic factors that are essential in the development, maintenance and survival of neurons (Vasina et al., 2006). Neurotrophic factors such as nerve growth factor (NGF), brain derived neurotrophic factor (BDNF), neurotrophin-3 (NT-3) and glial derived neurotrophic factor (GDNF) can stimulate axonal regeneration or sprouting after spinal cord injury (Ogawa et al., 2002; Lu et al., 2003, 2004; Llado et al., 2004). In the adult gastrointestinal tract, EG secrete various neurotrophic factors such as NGF, BDNF, NT-3 and GDNF, which are involved in a broad spectrum of physiological effects (von Boyen and Steinkamp, 2006). Moreover, inflammation increases release of NGF and NT-3 by enteric glia (Blennerhassett et al., 1996; von Boyen et al., 2002). In the present study, the inventors found that NGF and BDNF are secreted from cultured EG over time after isolation from the small intestine of adult rats. Therefore, it is likely that EG transplanted into injured spinal cords induce functional and histological improvement at least in part by releasing neurotrophic factors.

### Example 2

### Isolation, Purification, and Growth Enteric Glial Cells from Both Human Myenteric and Submucosal Plexus and Taenia Coli

The inventors have obtained the Research Ethics Board (REB) approval so that they can use intestine from humans taken at surgery for other purposes.

The inventors have successfully set up the conditions for culturing the human enteric glia and successfully isolated human enteric glia. They used DMEM/F12 medium supplemented with 1% penicillin/streptomycin and 10% foetal calf serum, rather than DMEM supplemented with 10% FCS, insulin, selenium, holo-transferrin and cortisone and to increase incubation time with dispase from one hour to one hour and twenty minutes.

To isolate enteric glia from a section of bowel requires abdominal surgery, and resection of a piece of bowel. If this technique were used in humans with spinal cord injury it would have potential complications, associated with surgical morbidity and, potentially, even mortality. However, enteric glia also exist in teniae coli—small vestigial attachments to the surface of the bowel—that can easily be removed using laparoscopic surgery without cutting into the lumen of the bowel. Teniae coli can be removed easily from the surface of the bowel though laparoscopic surgery. This much less invasive surgery is a huge advantage since one objective is to use the techniques developed in this research in human patients with spinal cord injury.

To extract enteric glia from human taenia coli the inventors used a modification of the extraction protocol the inventors had used in the guinea pig. Briefly, the caecum was excised at the iliocaecal and caecocolic junctions, rinsed multiple times in sterile PBS containing antibiotics to remove intestinal contents. The tissue was kept in PBS throughout the dissection. Under a dissecting microscope, the band was gently separated from the intestinal surface using the forceps and scalpel. Once dissected, the inventors were left with a band of mixed muscle, connective tissue and myenteric plexus. The inventors treated this with collagenase, an enzyme that digests the connective tissue holding the plexus and the muscle together, at the concentration, temperature and time described by Kimball and Mulholland et al., (J. Neurochem. 66, 604-612, 1996; Garrido, R. et al., J. Neurochem. 83, 556-564,2002). This separated the glial-containing plexus from its smooth muscle surroundings. However, the inventors found that using these conditions reduced the band being to a homogeneous slurry, from which it was impossible to extract pure myenteric plexus. To remove contaminating muscle from the plexus, it is necessary to distinguish the two—a feat possible if the digestion is arrested just after the plexus has been released from the band but before connections holding the smooth muscle begin to be digested. The inventors empirically determined an appropriate incubation period and ascertained that 12 hours at 4° C. followed by 25 minutes at 37° C. resulted in the ethereal, cloud-like plexus being freed from dense, compacted bands of muscle. After carefully removing the muscle from the mixture and being left with purified myenteric plexus, the inventors proceeded following the isolation procedures described before (Middlemiss et al., 2002; Jiang et al. 2003c).

However, after discovering that connective tissue was not sufficiently digested with this protocol, the inventors increased the time tissue was incubated in dispase from one hour to three hours.

Once presumptive EG had been obtained from either small intestinal myenteric plexuses or taenia coli, they were processed for immunostaining with GFAP following the procedures described before (Middlemiss et al., 2002).

### Results and Conclusion:

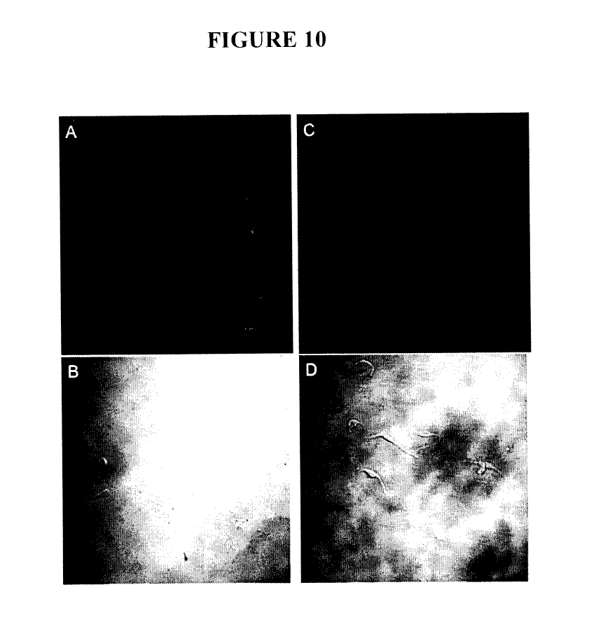

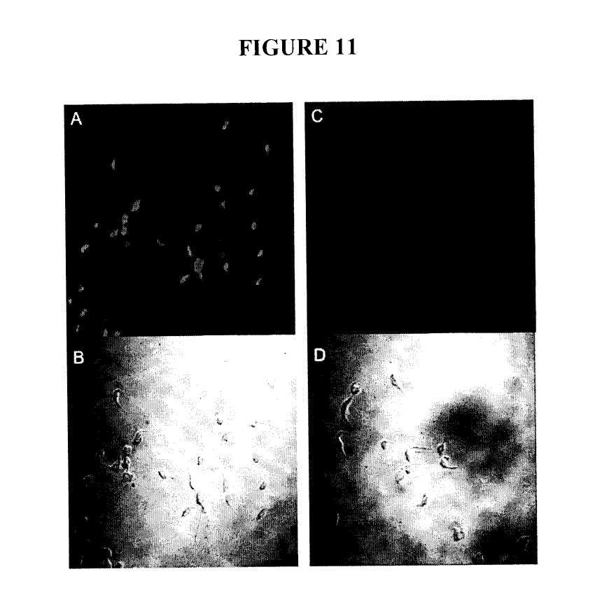

The result shows that both human myenteric and submucosal plexus-derived and human taenia coli-derived cells were GFAP positive staining. However, when primary antibodies were omitted in the cell medium the cells the specific staining of GFAP was entirely absent (FIGS. 10 and 11). Data indicates that the inventors have successfully isolated enteric glia from both human small intestine and taenia coli. This report is novel.

### Example 3

### Examine Effects of EG on Neurite Outgrowth of Dorsal Root Ganglian (DRG)

Neurotrophic factors promote neurite outgrowth (the beginning of axon or dendrite formation) and neuronal survival (for review, see Arévalo and Wu, Cell Molec. Life Sciences 63: 1523-1537, 2006). To determine whether EG-induced beneficial effect on axonal regeneration observed in vivo, the inventors examined the effect of EG-condition medium on neural growth of neurons from dorsal root ganglia, and ability of EG to stimulate regenerative neurite outgrowth with or without inhibitors to neurotrophic factors.

Dorsal root ganglia were extracted and cultured as per Hall (2006). Briefly, 8-16 week old rats were deeply anaesthetized with pentobarbital. A longitudinal incision was made along the spine. Tissue and vertebrae were removed to expose spinal cord. The cord was gently pushed aside and dorsal root ganglia were cut away from the nervous system and placed into ice-cold HBSS. Ganglia were then de-sheathed and incubated in a collagenase/dispase solution to digest connective tissue. Dissociated ganglia were triturated in bovine serum albumin (BSA)/PBS and resuspended in supplemented neurobasal medium (Neurobasal medium (Invitrogen), B-27 supplement (Invitrogen), L-glutamine (Invitrogen) and 1% penicillin/streptomycin (Invitrogen)) without nerve growth factor (NGF). Cells were pre-plated for 3.5 hours in 10 mL neurobasal medium to a 100 mm Petri dish to remove excess glia. Remaining floating cells were collected, centrifuged, counted and plated to wells of a poly-L-lysine and laminin-coated 24-well plate at 10,000 cells/well. For one experiment extra cells were available; these were seeded to uncoated wells. Half of the medium was changed every other day. Cells were allowed to incubate on the plate for three to five days before exposure to EG-conditioned medium.

After the acclimatization period, extracts were incubated with one of a) normal supplemented neurobasal medium, b) EG-conditioned neurobasal medium (see below). In some experiments NGF was added to either supplemented neurobasal or EG-conditioned neurobasal medium at 25 ng/mL final concentration. In some experiments 1 μg/mL goat anti-rat β-NGF (R&D Systems Cat. No. AF-556-NA) was added to supplemented neurobasal or EG-conditioned neurobasal medium.

EG-conditioned medium was prepared as follows. EG isolated from Wistar rat myenteric plexuses, passage 5-8 were seeded at 2×10⁴ cells/well to wells of a 6-well plate in DMEM/F12+10% FCS+1% penicillin/streptomycin (P/S) and incubated for 24 hours. Medium was removed and cells were rinsed with PBS. PBS was removed and replaced with DMEM/F12+2% FCS+1% P/S. Cells were incubated for 24 hours. After this incubation period medium was removed, cells rinsed with PBS, and then incubated for 24 hours in supplemented neurobasal medium. After the 24-hour period the supplemented neurobasal medium was considered to be conditioned. Conditioned medium was centrifuged at 1000 g for 4 minutes to remove any debris or floating cells.

A neurite was defined as a process of a neuron that extends at least one cell body diameter from the cell that has a small swelling, the growth cone, at its tip. Neurites on twenty or forty neurons per well of a twenty-four well plate were manually counted with a Nikon Diaphot microscope.

### Results and Conclusion:

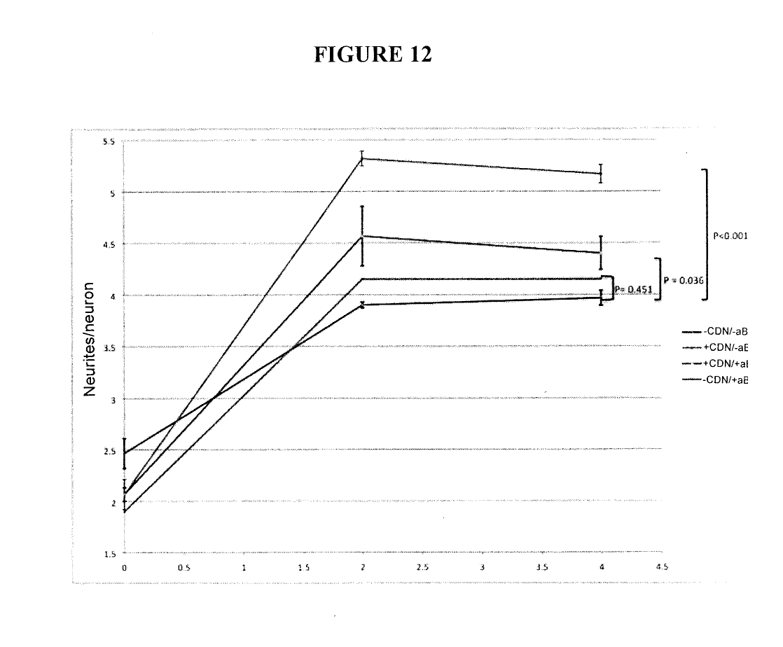

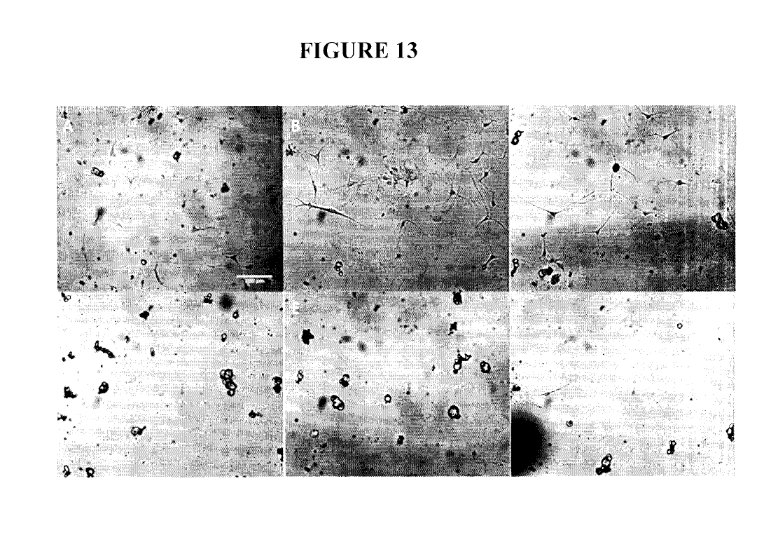

Data are shown in Table 1, 2 and FIGS. 12 and 13. Briefly, a two-way, repeated measures ANOVA was performed examining the effects of EG-conditioned medium with or without the addition of a neutralizing antibody to β-NGF. The inventors determined that EG-conditioned medium significantly enhances neurite outgrowth of DRG neurons compared with controlled group (df=1, F=25.880, P=0.02), while addition of anti-NGF neutralizing antibody reduced the effect of EG-conditioned medium. The data indicates that EG stimulated neurite outgrowth partially through NGF-mediated pathways. Since anti-NGF did entirely block the effect of EG on neurite outgrowth the inventors hypothesis that there are other pathways involved this process. Further studies are under investigation on exploring the effects of other neurotrophic factors on neurite outgrowth.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### ABBREVIATIONS

BBB Basso-Beattie-Bresnahan locomotor rating scale
BDNF brain derived neurotrophic factor
CNS central nervous system
CTM cutaneous trunci muscle
DMEM Dulbecco's modified eagle medium
EG enteric glia
FBS fetal bovine serum
FITC fluorescein isothiocyanate
FOR foot orienting response
GAP growth-associated protein
GDNF glial derived neurotrophic factor
GFAP glial fibrillary acidic protein
H&E hematoxylin and eosin
HLPR hind limb placing response
IPT inclined plane test
NGF neuron growth factor
NT-3 neurotrophin-3
OFWT open field walking test
PBS phosphate buffered saline
PFA paraformaldehyde
PNS peripheral nervous system
SCI spinal cord injury

TABLE 1
Role of neurotrophic factors in EG-mediated neurorestoration
Effect of EG-conditioned medium with or without
NGF on neurite number

| | (−)NGF | (−)NGF | (+)NGF | (+)NGF |
|---|---|---|---|---|
| | (−)CDN | (+)CDN | (−)CDN | (+)CDN |
| Day 1 | 0.175 | 1.175 | 2.325 | 3.3 |
| Day 3 | 1.65 | 4.5 | 5.65 | 6.3 |
| Day 5* | 5.425 | 7.425 | 7.675 | 10.5 |

Average number of neurites/neuron counted from non-aggregated dorsal root ganglionic neurons (40 neurons per well) that were plated with (+NGF) or without (−NGF) 25 ng/mL NGF in neurobasal medium that was (+CDN) or was not (−CDN) conditioned by enteric glia. *only 15 neurons could be counted in the +NGF/+CDN well at Day 5. There was one well for each experimental group. Neurites were first counted three days after extraction.

TABLE 2

| | (−) CDN | (+) CDN | (+) CDN | (−) CDN | (−) CDN | (−) CDN |
|---|---|---|---|---|---|---|
| | (−) αB | (−) αB | (+) αB | (+) αB | (−) αB | (−) αB |
| | (+) PC | (+) PC | (+) PC | (+) PC | (−) PC | (−) PC |
| Day -1* | 2.46 | 2.07 | 2.07 | 1.9 | 0.4 | 0.3 |
| Day 1 | 3.9 | 5.32 | 4.57 | 4.15 | 0.6 | 1.25 |
| Day 3 | 3.97 | 5.17 | 4.4 | 4.15 | 1 | 1.75 |

Average number of neurites/neuron counted from non-aggregated dorsal root ganglionic neurons (20 neurons per well) that were plated in medium that was (+CDN) or was not (−CDN) conditioned by enteric glia on wells that were (+PC) or were not (−PC) coated with poly-L-lysine and laminin. *A baseline count made one day before conditioned medium was added. There were three wells for the ±CDN/−αB/+PC and +CDN/+αB/+PC groups. There was one well for each of the other experimental groups. Baseline count was made three days after extraction.

### FULL CITATIONS FOR REFERENCES REFERRED TO IN THE SPECIFICATION

1. Abbott N J, Ronnback L, Hansson E (2006) Astrocyte-endothelial interactions at the blood-brain barrier. Nat Rev Neurosci 7, 41-53. Review.
2. Arévalo, J. C. and Wu, S. H. (2006) Neurotrophin signaling: many exciting surprises! Cell. Mol. Life Sci. 63, 1523-1537
3. Barber P C, Lindsay R M (1982) Schwann cells of the olfactory nerves contain glial fibrillary acidic protein and resemble astrocytes. Neuroscience 7, 3077-3090.
4. Barnett S C, Riddell J S (2007) Olfactory ensheathing cell transplantation as a strategy for spinal cord repair—what can it achieve? Nat Clin Pract Neurol 3, 152-161.
5. Barrett C P, Donati E J, Guth L (1984) Differences between adult and neonatal rats in their astroglial response to spinal injury. Exp Neurol 84, 374-385.
6. Basso D M, Beattie M S, Bresnahan J C (1995) A sensitive and reliable locomotor rating scale for open field testing in rats. J Neurotrauma 12, 1-21.
7. Basso D M, Beattie M S, Bresnahan J C (1996) Graded histological and locomotor outcomes after spinal cord contusion using the NYU weight-drop device versus transection. Exp Neurol 139, 244-256.
8. Basso D M (2000) Neuroanatomical substrates of functional recovery after experimental spinal cord injury: implications of basic science research for human spinal cord injury. Phys Ther 80, 808-817.
9. Bechmann I, Galea I, Perry V H (2007) What is the blood-brain barrier (not)? Trends Immunol 28, 5-11.
10. Bishop, A. E. et al. (1985) Combined Immunostaining of Neurofilaments, Neuron Specific Enolase, Gfap and S-100—a Possible Means for Assessing the Morphological and Functional Status of the Enteric Nervous System. Histochemistry 82, 93-97.
11. Blennerhassett M G, Seaton B, Hsuch B, Lamb D P (1996) Neurotrophin production in inflamed rat intestine. Gastroenterology 110, A1060.
12. Blight A R (1991) Morphometric analysis of a model of spinal cord injury in guinea pigs, with behavioral evidence of delayed secondary pathology. J Neurol Sci 103:156-171.
13. Bresnahan J C, Beattie M S, Todd F D III, Noyes D H (1987) A behavioral and anatomical analysis of spinal cord injury produced by a feedback-controlled impaction device. Exp Neurol 95, 548-570.
14. Bundesen L Q, Scheel T A, Bregman B S, Kromer L F (2003) Ephrin-B2 and EphB2 regulation of astrocyte-meningeal fibroblast interactions in response to spinal cord lesions in adult rats. J Neurosci 23, 7789-7800.
15. Bunge R P, Puckett W R, Hiester R D (1997) Observations on the pathology of several types of human spinal cord injury, with emphasis on the astrocyte response to penetrating injuries. Adv Neurol 72, 305-315.
16. Cabarrocas J, Savidge T C, Liblau R S (2003) Role of enteric glial cells in inflammatory bowel disease. Glia 41, 81-93.
17. Doucette R (1990) Glial influences on axonal growth in the primary olfactory system. Glia 3, 433-449.
18. Dumont R J, Verma S, Okonkwo D O, Hurlbert R J, Boulos P T, Ellegala D B, Dumont A S (2001) Acute spinal cord injury, part I: pathophysiologic mechanisms. Clin Neuropharmacol 24, 254-264.
19. Ferri G L, Probert L, Cocchia D, Michetti F, Marangos P J, Polak J M (1982) Evidence for the presence of S-100 protein in the glial component of the human enteric nervous system. Nature 297, 409-410.
20. Filbin M T (2003) Myelin-associated inhibitors of axonal regeneration in the adult mammalian CNS. Nat Rev Neurosci 4, 703-713.
21. Frisen J, Haegerstrand A, Risling M, Fried K, Johansson C B, Hammarberg H, Elde R, Hokfelt T, Cullheim S (1995). Spinal axons in central nervous system scar tissue are closely related to laminin-immunoreactive astrocytes. Neuroscience 65, 293-304.
22. Garrido, R. et al. (2002) Presence of functionally active protease-activated receptors 1 and 2 in myenteric glia. J. Neurochem. 83, 556-564.
23. Georgiou, J. and Charlton, M. P. (1999) Non-myelin-forming perisynaptic schwann cells express protein zero and myelin-associated glycoprotein. Glia 27, 101-109
24. Gruner J A, Yee A K, Blight A R (1996) Histological and functional evaluation of experimental spinal cord injury: evidence of a stepwise response to graded compression. Brain Res 729, 90-101.
25. Hall, A. K. (2006) Rodent sensory neuron culture and analysis. Curr. Protoc. Neurosci. Chapter 3, Unit 3.19.
26. Jaeger C B, Toombs J P, Borgens R B (1993) Grafting in acute spinal cord injury: morphological and immunological aspects of transplanted adult rat enteric ganglia. Neuroscience 52, 333-346.
27. Jessen K R, Mirsky R (1980) Glial cells in the enteric nervous system contain glial fibrillary acidic protein. Nature 286, 736-737.
28. Jessen K R, Mirsky R (1983) Astrocyte-like glia in the peripheral nervous system: an immunohistochemical study of enteric glia. J Neurosci 3, 2206-2218.
29. Jiang S, Wang J, Khan M I, Middlemiss P J, Salgado-Ceballos H, Werstiuk E S, Wickson R, Rathbone M P (2003a) Enteric glia promote regeneration of transected dorsal root axons into spinal cord of adult rats. Exp Neurol 181, 79-83.
30. Jiang S, Khan M I, Wang J, Middlemiss P J, Werstiuk E S, Wickson R, Rathbone M P (2003b) Enteric glia promote functional recovery of CTM reflex after dorsal root transection. Neuroreport 14, 1301-1304.
31. Jiang S, Khan M I, Lu Y, Wang J, Buttigieg J, Werstiuk E S, Ciccarelli R, Caciagli F, Rathbone M P (2003c) Guanosine promotes myelination and functional recovery in chronic spinal injury. Neuroreport 14, 2463-2467.
32. Jiang S, Khan M I, Middlemiss P J, Lu Y, Werstiuk E S, Crocker C E, Ciccarelli R, Caciagli F, Rathbone M P (2004) AIT-082 and methylprednisolone singly, but not in combination, enhance functional and histological improvement after acute spinal cord injury in rats. Int J Immunopathol Pharmacol 17, 353-366.
33. Jiang S, Khan M I, Lu Y, Werstiuk E S, Rathbone M P (2005) Acceleration of blood-brain barrier formation after transplantation of enteric glia into spinal cords of rats. Exp Brain Res 162, 56-62.
34. Kerasidis H, Wrathall J R, Gale K (1987) Behavioral assessment of functional deficit in rats with contusive spinal cord injury. J Neurosci Methods 20, 167-179.
35. Kimball, B. C. and Mulholland, M. W. (1996) Enteric glia exhibit P2U receptors that increase cytosolic calcium by a phospholipase C-dependent mechanism. J. Neurochem. 66, 604-612.
36. Lawrence J M, Raisman G, Mirsky R, Jessen K R (1991) Transplantation of postnatal rat enteric ganglia into denervated adult rat hippocampus. Neuroscience 44, 371-379.
37. Llado J, Haenggeli C, Maragakis N J, Snyder E Y, Rothstein J D (2004) Neural stem cells protect against glutamate-induced excitotoxicity and promote survival of injured motor neurons through the secretion of neurotrophic factors. Mol Cell Neurosci 27, 322-331.
38. Lu P, Jones L L, Snyder E Y, Tuszynski M H (2003) Neural stem cells constitutively secrete neurotrophic factors and promote extensive host axonal growth after spinal cord injury. Exp Neurol 181, 115-129.
39. Lu P, Yang H, Jones L L, Filbin M T, Tuszynski M H (2004) Combinatorial therapy with neurotrophins and cAMP promotes axonal regeneration beyond sites of spinal cord injury. J Neurosci 24, 6402-6409.
40. Lu P, Jones L L, Tuszynski M H (2007) Axon regeneration through scars and into sites of chronic spinal cord injury. Exp Neurol 203, 8-21.
41. Middlemiss P, Jiang S, Wang J, Rathbone M P (2002) A method for purifying enteric glia from rat myenteric plexus. In Vitro Cell Dev Biol Anim 38, 188-190.
42. Neunlist M, Aubert P, Bonnaud S, Van Landeghem L, Coron E, Wedel T, Naveilhan P, Ruhl A, Lardeux B, Savidge T, Paris F, Galmiche J P (2007) Enteric glia inhibit intestinal epithelial cell proliferation partly through a TGF-beta1-dependent pathway. Am J Physiol Gastrointest Liver Physiol 292, G231-G241.
43. Ogawa Y, Sawamoto K, Miyata T, Miyao S, Watanabe M, Nakamura M, Bregman B S, Koike M, Uchiyama Y, Toyama Y, Okano H (2002) Transplantation of in vitro-expanded fetal neural progenitor cells results in neurogenesis and functional recovery after spinal cord contusion injury in adult rats. J Neurosci Res 69, 925-933.
44. Pearse D D, Sanchez A R, Pereira F C, Andrade C M, Puzis R, Pressman Y, Golden K, Kitay B M, Blits B, Wood P M, Bunge M B (2007) Transplantation of Schwann cells and/or olfactory ensheathing glia into the contused spinal cord: Survival, migration, axon association, and functional recovery. Glia 55, 976-1000.
45. Pixley S K (1992) The olfactory nerve contains two populations of glia, identified both in vivo and in vitro. Glia 5, 269-284.
46. Reier P (2004) Cellular transplantation strategies for spinal cord injury and translational neurobiology. NeuroRx 1, 424-451.
47. Richter M W, Roskams A J (2007) Olfactory ensheathing cell transplantation following spinal cord injury: Hype or hope? Exp Neurol June 30; [Epub ahead of print]
48. Rivlin A S, Tator C H (1977) Objective clinical assessment of motor function after experimental spinal cord injury in the rat. J Neurosurg 47, 577-581.
49. Ruhl A (2005) Glial cells in the gut. Neurogastroenterol Motil 17, 777-790.
50. Savidge T C, Newman P, Pothoulakis C, Ruhl A, Neunlist M, Bourreille A, Hurst R, Sofroniew M V (2007a) Enteric glia regulate intestinal barrier function and inflammation via release of S-nitrosoglutathione. Gastroenterology 132, 1344-1358.
51. Savidge T C, Sofroniew M V, Neunlist M (2007b) Starring roles for astroglia in barrier pathologies of gut and brain. Lab Invest 87, 731-736.
46. Schwab M E (2000) Neurobiology: finding the lost target. Nature 403, 257-260.
52. Silver J, Miller J H (2004) Regeneration beyond the glial scar. Nat Rev Neurosci 5, 146-156.
53. Tew E M M, Anderson P N, Burnstock G (1992) Implantation of the myenteric plexus into the corpus striatum of adult rats: survival of the neurons and glia and interactions with host brain. Restorative Neurol. Neuroscience 4, 311-321.
54. Tew E M, Saffrey M J, Anderson P N, Burnstock G (1993) Postnatal rat NADPH-diaphorase-containing myenteric neurons extend processes when transplanted into adult rat corpus striatum. Exp Neurol 124, 265-273.
55. Tew E M, Patrick N A, Saffrey M J, Burnstock G (1994) Transplantation of the postnatal rat myenteric plexus into the adult rat corpus striatum: an electron microscopic study. Exp Neurol 129,120-129.
56. Vasina V, Barbara G, Talamonti L, Stanghellini V, Corinaldesi R, Tonini M, De Ponti F, De Giorgio R (2006) Enteric neuroplasticity evoked by inflammation. Auton Neurosci 126-127, 264-272.
57. von Boyen G B, Reinshagen M, Steinkamp M, Adler G, Kirsch J (2002). Gut inflammation modulated by the enteric nervous system and neurotrophic factors. Scand J Gastroenterol 37, 621-625.
58. von Boyen G B, Steinkamp M (2006) The enteric glia and neurotrophic factors. Z Gastroenterol 44, 985-990.
59. von Boyen G B, Steinkamp M, Geerling I, Reinshagen M, Schafer K H, Adler G, Kirsch J (2006a) Proinflammatory cytokines induce neurotrophic factor expression in enteric glia: a key to the regulation of epithelial apoptosis in Crohn's disease. Inflamm Bowel Dis 12, 346-354.
60. von Boyen G B, Steinkamp M, Reinshagen M, Schafer K H, Adler G, Kirsch J (2006b) Nerve growth factor secretion in cultured enteric glia cells is modulated by proinflammatory cytokines. J Neuroendocrinol 18, 820-825.

We claim:

1. A use of an enteric glial cell for reducing tissue damage in the nervous system of an animal.

2. A use according to claim 1 wherein an enteric glial cells are autologous.

3. A use according to claim 1 or 2 wherein the animal has a spinal cord injury.

4. A use according to any one of claims 1-3 wherein cystic lesions are reduced.

5. A use according to any one of claims 1 to 4 wherein the sprouting of GAP-43 axons is induced.

6. A use of an enteric glial cell for improving locomotor function in an animal with a nerve injury.

7. A use according to claim 6 wherein an enteric glial cells are autologous.

8. A use according to claim 6 or 7 wherein the animal has a spinal cord injury.

9. The use according to any one of claims 1 to 8 wherein the animal is a human.

10. The use according to claim 9 wherein the enteric glial cells are obtained from the small intestine or taenia coli of the human.
